SEQUENCE LISTING (2 sequences)

SEQ ID NO: 1 - DNA, 1245 bp, Homo sapiens
  source 1..1245: mol_type = genomic DNA
cttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcattacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccacc

SEQ ID NO: 2 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

(19) United States
(12) Patent Application Publication
(10) Pub. No.: US 2026/0287591 A1
(43) Pub. Date: Sep. 24, 2026

(54) METHOD FOR DISCRIMINATING MONO-IMMUNOTHERAPY FROM COMBINED IMMUNOTHERAPY IN CANCERS

(71) Applicants: INSTITUT NATIONAL DE LA SANTÉ ET DE LA RECHERCHE MÉDICALE, Paris (FR); UNIVERSITÉ PARIS CITÉ, Paris (FR); ASSISTANCE PUBLIQUE-HÔPITAUX DE PARIS (APHP), Paris (FR)

(72) Inventors: Eric TARTOUR, Paris (FR); Céleste LEBBÉ, Paris (FR); Stéphane OUDARD, Paris (FR); Ikuan SAM, Paris (FR); Nadine BEN HAMOUDA, Paris (FR)

(21) Appl. No.: 19/168,578

(22) PCT Filed: Mar. 27, 2024
(86) PCT No.: PCT/EP2024/058363

§ 371 Date: Sep. 24, 2025

(30) Foreign Application Priority Data
Mar. 28, 2023 (EP) 23305423.8
Apr. 26, 2023 (EP) 23315114.1

(51) Int. Cl. G01N 33/575; A61K 39/00; A61K 39/395; A61P 35/04; C07K 16/28

(52) U.S. Cl. CPC G01N 33/5758; A61K 39/3955; A61P 35/04; C07K 16/2818; A61K 2039/505; A61K 2039/507; C07K 2317/21; G01N 2333/70578

(57) ABSTRACT

Combination therapy with anti-PD-1 and anti-CTLA-4 is now indicated in many cancers. It appears to be more effective than anti-PD-1 monotherapy, but more toxic. This work has shown that plasma levels of soluble CD27 are inversely correlated with survival in melanoma patients treated with anti-PD-1. This result was validated in 2 independent cohorts of patients representing 180 patients in total. Interestingly, this predicting role of sCD27 in anti-PD-1 treated melanoma patients was found both with an optimal cut-off defined by the Youden test, but also with the same cut-off previously defined in kidney cancer. This suggests some analytical robustness of this marker. Inventors have also shown that plasma sCD27 levels prior to anti-PD-1 treatment are inversely correlated with progression free survival (PFS). On the contrary, in patients with metastatic melanoma treated with anti-PD-1 and anti-CTLA-4, plasma sCD27 levels are not statistically significantly associated with patient survival or progression-free survival. They therefore find a discrepancy between the predictive impact of sCD27 on survival of patients treated with anti-PD-1 immunotherapy alone or with combined anti-PD-1 and anti-CTLA-4 immunotherapy. Plasma sCD27 concentrations represents a new type of biomarker to help in the management of these patients.

### FIELD OF THE INVENTION

The invention is in the field of oncology. More particularly, the invention relates to method for discriminating monotherapy from combined therapy in melanoma or kidney cancer.

### BACKGROUND OF THE INVENTION

Although melanoma has the highest response rates to immunotherapies compared to other cancers, a considerable proportion of patients do not benefit and are exposed to adverse effects and even hyperprogression. More and more immunotherapies are being used in the first line treatment of metastatic or locally advanced melanoma, but also in the perioperative treatment of resectable melanoma (Table 1). At the metastatic stage, the recommendation is to choose immunotherapy as a general rule, even in patients with BRAF-mutated melanoma, but the choice between anti-PD1 monotherapy and double immunotherapy with anti-PD1 combined with anti-CTLA-4, which is more effective but more toxic, is currently guided solely by clinical arguments (age, comorbidities). At resected stage III, a combination of targeted therapies (anti BRAF and anti MEK) for BRAF mutated melanomas and two anti-PD1 are approved as adjuvants [1]. In addition, different immunotherapy treatments are becoming available without always having comparative data with a high level of evidence to choose the optimal option for the eligible patient. In these situations, biomarkers that could guide the different therapeutic options for the same indication and avoid toxic combination therapies in the absence of predicted resistance to a monotherapy would improve patient management.

Different types of biomarkers have been reported but none are currently used for patient management.

### SUMMARY OF THE INVENTION

The invention relates to a method for discriminating a monotherapy with an immune check-point inhibitor for a bi-therapy with a combination of immune-checkpoint inhibitor for a subject suffering from a cancer comprising: i) quantifying the level of soluble CD27 (sCD27) in a biological sample obtained from the subject before a treatment with a monotherapy or bi-therapy, ii) comparing the level of sCD27 quantified at step i) with its corresponding predetermined reference values and iii) concluding that the monotherapy will be chosen when the level of sCD27 quantified at step i) is lower than its corresponding predetermined reference value or concluding that the bi-therapy will be chosen when the level of sCD27 quantified at step i) is higher than its corresponding predetermined reference value.

In particular, the invention is defined by claims.

### DETAILED DESCRIPTION OF THE INVENTION

Combination therapy with anti-PD-1 and anti-CTLA-4 is now indicated in many cancers. It appears to be more effective than anti-PD-1 monotherapy, but more toxic.

This work has shown that plasma levels of soluble CD27 are inversely correlated with survival in melanoma patients treated with anti-PD-1. This result was validated in 2 independent cohorts of patients representing 180 patients in total. Interestingly, this predicting role of sCD27 in anti-PD-1 treated melanoma patients was found both with an optimal cut-off defined by the Youden test, but also with the same cut-off previously defined in kidney cancer. This suggests some analytical robustness of this marker. Inventors have also shown that plasma sCD27 levels prior to anti-PD-1 treatment are inversely correlated with progression free survival (PFS). On the contrary, in patients with metastatic melanoma treated with anti-PD-1 and anti-CTLA-4, plasma sCD27 levels are not statistically significantly associated with patient survival or progression-free survival. They therefore find a discrepancy between the predictive impact of sCD27 on survival of patients treated with anti-PD-1 immunotherapy alone or with combined anti-PD-1 and anti-CTLA-4 immunotherapy. Plasma sCD27 concentrations represents a new type of biomarker to help in the management of these patients and thus to avoid to use a combination therapy with toxic effects.

Method for Discriminating Mono-Immunotherapy from Combined Therapy

Accordingly, in a first aspect, the invention relates to a method for discriminating a monotherapy with an immune check-point inhibitor form a bi-therapy with a combination of immune-checkpoint inhibitor for a subject suffering from a cancer comprising: i) quantifying the level of soluble CD27 (sCD27) in a biological sample obtained from the subject before a treatment with a monotherapy or bi-therapy, ii) comparing the level of sCD27 quantified at step i) with its corresponding predetermined reference values and iii) concluding that the monotherapy will be chosen when the level of sCD27 quantified at step i) is lower than its corresponding predetermined reference value or concluding that the bi-therapy will be chosen when the level of sCD27 quantified at step i) is higher than its corresponding predetermined reference value.

As used herein, the term “CD27” is a member of the tumor necrosis factor receptor superfamily. It is currently of interest to immunologists as a co-stimulatory immune checkpoint molecule. CD27 binds to ligand CD70, and plays a key role in regulating B-cell activation and immunoglobulin synthesis. A soluble form of CD27 (sCD27), a 32-kD protein identical to the extracellular domain of membrane-bound CD27, can be released after lymphocyte activation by differential splicing of the receptor protein or shedding from the cell surface by proteases.

The human CD27 has the following nucleotide sequence in the art SEQ ID NO:1:

| | |
|---|---|
| 1 | cttcaaaggt tggcttgcca cctgaagcag ccactgccca |
| | gggggtgcaa agaagagaca |
| 61 | gcagcgccca gcttggaggt gctaactcca gaggccagca |
| | tcagcaactg ggcacagaaa |
| 121 | ggagccgcct gggcagggac catggcacgg ccacatccct |
| | ggtggctgtg cgttctgggg |
| 181 | accctggtgg ggctctcagc tactccagcc cccaagagct |
| | gcccagagag gcactactgg |
| 241 | gctcagggaa agctgtgctg ccagatgtgt gagccaggaa |
| | cattcctcgt gaaggactgt |
| 301 | gaccagcata gaaaggctgc tcagtgtgat ccttgcatac |
| | cgggggtctc cttctctcct |
| 361 | gaccaccaca cccggcccca ctgtgagagc tgtcggcact |
| | gtaactctgg tcttctcgtt |
| 421 | cgcaactgca ccatcactgc caatgctgag tgtgcctgtc |
| | gcaatggctg gcagtgcagg |
| 481 | gacaaggagt gcaccgagty tgatcctctt ccaaaccctt |
| | cgctgaccgc tcggtcgtct |
| 541 | caggccctga gcccacaccc tcagcccacc cacttacctt |
| | atgtcagtga gatgctggag |
| 601 | gccaggacag ctgggcacat gcagactctg gctgacttca |
| | ggcagctgcc tgcccggact |
| 661 | ctctctaccc actggccacc ccaaagatcc ctgtgcagct |
| | ccgattttat tcgcatcctt |
| 721 | gtgatcttct ctggaatgtt ccttgttttc accctggccg |
| | gggccctgtt cctccatcaa |
| 781 | cgaaggaaat atagatcaaa caaaggagaa agtcctgtgg |
| | agcctgcaga gccttgtcat |
| 841 | tacagctgcc ccagggagga ggagggcagc accatcccca |
| | tccaggagga ttaccgaaaa |
| 901 | ccggagcctg cctgctcccc ctgagccagc acctgcggga |
| | gctgcactac agccctggcc |
| 961 | tccaccccca ccccgccgac catccaaggg agagtgagac |
| | ctggcagcca caactgcagt |
| 1021 | cccatcctct tgtcagggcc ctttcctgtg tacacgtgac |
| | agagtgcctt ttcgagactg |
| 1081 | gcagggacga ggacaaatat ggatgaggtg gagagtggga |
| | agcaggagcc cagccagctg |
| 1141 | cgcctgcgct gcaggagggc gggggctctg gttgtaaaac |
| | acacttcctg ctgcgaaaga |
| 1201 | cccacatgct acaagacggg caaaataaag tgacagatga |
| | ccacc |

The human CD27 has the following amino acid sequence in the art SEQ ID NO:2:

| | |
|---|---|
| 1 | marphpwwlc vlgtlvglsa tpapkscper hywaqgklcc |
| | qmcepgtflv kdcdqhrkaa |
| 61 | qcdpcipgvs fspdhhtrph cescrhcnsg llvrnctita |
| | naecacrngw qcrdkectec |
| 121 | dplpnpslta rssqalsphp qpthlpyvse mleartaghm |
| | qtladfrqlp artlsthwpp |
| 181 | qrslcssdfi rilvifsgmf lvftlagalf lhqrrkyrsn |
| | kgespvepae pchyscpree |
| 241 | egstipiqed yrkpepacsp |

As used herein, the term “subject” denotes a mammal, such as a rodent, a feline, a canine, and a primate. Particularly, the subject according to the invention is a human. More particularly, the subject according to the invention has or is susceptible to have a cancer expressing CD70. In a particular embodiment, the subject according to the invention has or is susceptible to have a cancer as described above. In another embodiment, the subject according to the invention has or is susceptible to have metastatic cancer.

As used herein, the term “cancer” refers to a malignant growth or tumour resulting from an uncontrolled division of cells. The term “cancer” includes primary tumors and metastatic tumors.

In addition, the cancer may specifically be of the following histological type, though it is not limited to these: neoplasm, malignant; carcinoma; carcinoma undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia, pleural cancer or colorectal cancer.

In a particular embodiment, the cancer is melanoma.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye. In a particular embodiment, the melanoma is cutaneous melanoma.

In a particular embodiment, the cancer is the kidney cancer.

As used herein, the terms “kidney cancer,” “renal cancer,” or “renal cell carcinoma” used interchangeably and refer to cancer that has arisen from the kidney. The terms “renal cell cancer” or “renal cell carcinoma” (RCC), as used herein, refer to cancer which originates in the lining of the proximal convoluted tubule. More specifically, RCC encompasses several relatively common histologic subtypes: clear cell renal cell carcinoma, papillary (chromophil), chromophobe, collecting duct carcinoma, and medullary carcinoma. Clear cell renal cell carcinoma (ccRCC) is the most common subtype of RCC. In a particular embodiment, the cancer is a metastatic renal cell carcinoma.

In another embodiment, the cancer is lung cancer.

As used herein, the term “lung cancer” includes, but is not limited to all types of lung cancers at all stages of progression like lung carcinomas metastatic lung cancer, non-small cell lung carcinomas (NSCLC) such as lung adenocarcinoma, squamous cell carcinoma, or small cell lung carcinomas (SCLC). In some embodiments, the subject suffers from a non-small cell lung carcinoma (NSCLC).

In a particular embodiment, the cancer is resistant or metastatic cancer.

As used herein, the term “resistant cancer” also called as “metastatic cancer” refers to a cancer which does not respond to a treatment. The cancer may be resistant at the beginning of treatment, or it may become resistant during treatment. The resistance to drug leads to rapid progression of metastatic cancer.

As used herein, the term “biological sample” refers to any sample obtained from a subject, such as a serum sample, a plasma sample, a urine sample, a blood sample, a lymph sample, tumor sample or a tissue biopsy. In a particular embodiment, biological sample for the determination of an expression level includes samples such as a blood sample, a lymph sample, or a biopsy.

In a particular embodiment, the biological sample is a blood sample. In another embodiment, the biological sample is a plasma sample. Typically, cancer subjects and healthy donors were used to analyze sCD27 concentration by CD27 (Soluble) Human Instant ELISA Kit (ThermoFisher Scientific, Massachusetts, United States) according to the manufacturer's instructions. Data were acquired with MRX Revelation Microplate Reader (DYNEX Technologies, Virginia, United States).

As used herein, the term “level of soluble CD27” refers to the concentration of soluble CD27. Typically, the level or concentration of the soluble CD27 gene may be determined by any technology known by a person skilled in the art. In particular, the concentration may be measured at the genomic and/or nucleic and/or protein level. In a further embodiment, the soluble CD27 is characterized by Luminex, electrochemoluminescence or ultra-sensitive immunoassay (Simoa . . . ). Typically, the apoptosis, exhaustion, proliferation and cytotoxicity of lymphocytes T are measured by Flow cytometry by Single-cell RNA-seq, by in situ multiplex immunofluorescence and/or immunohistochemistry. In another embodiment, the production of cytokines and/or chemokines by lymphocytes T is measured by Luminex.

In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e.g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the amount of sCD27 present in the plasma sample is detected by mass spectrometry.

In some embodiments, a score which is a composite of the level of the soluble CD27 is determined and compared to a reference value.

When a low concentration of soluble CD27 than the reference value is determined, it is indicative that the subject will achieve a response to monotherapy treatment and thus he will have a high survival time.

When a high concentration of soluble CD27 than the reference value is determined, it is indicative that the subject will not achieve a response to monotherapy treatment, and thus it is recommended to use a bi-therapy with a combination of immune check point inhibitors (e.g anti-PD-1 and anti-CTLA-4).

In a particular embodiment, the predetermined reference value is 128.03 U/ml. Said predetermined reference value is determined in cancer patients by optimal cu-off value.

As used herein, the term “predetermined reference value” refers to a threshold value or a cut-off value, which can be determined experimentally, empirically, or theoretically. A threshold value can also be arbitrarily selected based upon the existing experimental and/or clinical conditions, as would be recognized by a person of ordinary skilled in the art. For example, retrospective measurement of the level of the soluble CD27 in properly banked historical plasma samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the soluble CD27 in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

As used herein, the term “immunotherapy” or “immunotherapy treatment” refers to a cancer therapeutic treatment using the immune system to reject cancer. The therapeutic treatment stimulates the patient's immune system to attack the malignant tumor cells. It includes immunization of the patient with tumor antigens (e.g. by administering a cancer vaccine), in which case the patient's own immune system is trained to recognize tumor cells as targets to be destroyed, or administration of molecules stimulating the immune system such as cytokines, or administration of therapeutic antibodies such as drugs, in which case the patient's immune system is recruited by the therapeutic antibodies to destroy tumor cells. In particular, antibodies are directed against specific antigens such as the unusual antigens that are presented on the surfaces of tumors.

As used herein, the term “mono-immunotherapy” refers to use of one immunotherapy treatment. In the context of the invention, the monotherapy is performed with an anti PD-1 (e.g. nivolumab) or an anti-PD-L1 inhibitor.

As used herein, the terms “bi-therapy”, “bi-immunotherapy”, “combined-immuno therapy” or “combined therapy” are used interchangeably and refer to use of two immunotherapy treatments (as a combined therapy). In the context of the invention, the bi-therapy (as a combined therapy) is performed with anti-PD-1 and anti-CTLA-4 inhibitor (e.g nivolumab and ipilimumab) or anti-PD-L1 and anti-CTLA-4 inhibitors or anti-PD-1 and anti-LAG-3 inhibitors or anti-PD-L1 and anti-LAG-3 inhibitors.

Thus, in other words the invention relates to a method for discriminating a monotherapy with an anti-PD1 or an anti-PD-L1 inhibitor from a bi-therapy with a combination selected from the group consisting of a) anti-PD1 and an anti-CTLA4 inhibitors, b) an anti-PD-L1 and anti-CTLA-4 inhibitors, c) anti-LAG-3 and anti-CTLA-4 inhibitors, or d) anti-LAG-3 and anti-CTLA4 inhibitors for a subject suffering from a cancer comprising: i) quantifying the level of soluble CD27 (sCD27) in a biological sample obtained from the subject before a treatment with a monotherapy or bi-therapy, ii) comparing the level of sCD27 quantified at step i) with its corresponding predetermined reference values and iii) concluding that the monotherapy will be chosen when the level of sCD27 quantified at step i) is lower than its corresponding predetermined reference value or concluding that the bi-therapy will be chosen when the level of sCD27 quantified at step i) is higher than its corresponding predetermined reference value.

As used herein, the term “immune checkpoint” or “immune checkpoint protein” has its general meaning in the art and refers to a molecule that is expressed by immune or cancer cells in that either turn up a signal (stimulatory checkpoint molecules) or turn down a signal (inhibitory checkpoint molecules). Many of the immune checkpoints are regulated by interactions between specific receptor and ligand pairs. Overexpression of inhibitory checkpoint molecules by cancer cells have often been associated with inhibition of anti tumor immune response as immune cell express their ligand or receptor counterparts.

Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

As used herein, the expressions “immune checkpoint inhibitor”, “checkpoint inhibitor” or “checkpoint blockade cancer immunotherapy agent” are used interchangeably and have its general meaning in the art and refers to any compound inhibiting the function of an immune checkpoint protein. Inhibition includes reduction of function and full blockade. The immune checkpoint inhibitors include peptides, proteins, antibodies, nucleic acid molecules and small molecules. Preferred immune checkpoint inhibitors are antibodies that specifically recognize immune checkpoint proteins. Examples of immune checkpoint inhibitors are provided here below under the associated paragraph.

In a particular embodiment, the immune checkpoint inhibitor is an antibody.

Typically, antibodies are directed against PD-1, CTLA-4, A2AR, B7-H3, B7-H4, BTLA, CD277, IDO, KIR, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In a particular embodiment, the anti-PD-1 or anti-PD-L1 antibody is atezolizumab, durvalumab, avelumab, nivolumab, pembrolizumab, pidilizumab, cemiplimab, camrelizumab, sintilimab (IB1I308), tislelizumab (BGB-A317), toripalimab (JS 001), dostarlimab (TSR-042, WBP-285), BMS 936559, MPDL3280A, MSB0010718C, MEDI4736 and any combination thereof.

In a particular embodiment, the immune checkpoint inhibitor is an antibody directed against CTLA-4. Antibodies directed against CTLA-4 are also known such as ipilimumab, tremelimumab, MK-1308, AGEN-1884, XmAb20717 (Xencor), MEDI5752 (AstraZeneca).

In some embodiments, the monotherapy is performed with an-anti PD-1. More particularly, the anti-PD-1 is nivolumab.

In some embodiments, the bi-therapy (as a combined therapy) is performed with anti-PD-1 inhibitor and anti-CTLA-4 inhibitor, and in particular with anti-PD-1 antibody and anti-CTLA-4 antibody. More particularly, the anti-PD-1 is nivolumab and anti-CTLA-4 is ipilimumab.

In some embodiments, the immune checkpoint inhibitor is an anti-PD-L2 antibody such as described in U.S. Pat. Nos. 7,709,214, 7,432,059 and 8,552,154.

In some embodiments, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In a particular embodiment, the immune checkpoint inhibitor is a monoclonal antibody directed against LAG-3. Antibodies directed against LAG-3 are also known such as: Relatlimab (BMS-986016), Favezelimab (MK-4280, Merck), LAG3-Ab (Protheragen), TJA-3(I-Mab), LBL-007(BeiGene), LAG525(Novartis), Tesaro (GSK) (TSR-033), Sym022 (Symphogen), GSK2831781 (GlaxoSmith), INCAGNO2385 (Incyte Biosciences International), IMP321(Prima BioMed/Immutep), MGDO13 (MacroGenics), FS118 (F-Star), R07247669 (Hoffmann-La Roche), EMB-02(Shanghai EpimAb Biotherapeutics), XmAb841 (Xencor), Fianlimab (REGN3767) or IB1I323(Innovent Biologics).

In a particular embodiment, the immune checkpoint inhibitor is a bispecific antibody directed against LAG-3 and another immune check point (PD-1, PDL-1, PD-2 etc). Bispecific antibodies directed against LAG-3 are also known such as: Tebotelimab (MGD013, Macrogenetics), FS118 (F-star Therapeutics), R07247669 (Hoffmann-La Roche), EMB-02 (EpimAb Biotherapeutics).

In some embodiments, the bi-therapy (as a combined therapy) is performed with anti-PD-1 and anti-LAG-3. More particularly, the anti-PD-1 is nivolumab and anti-LAG-3 is Relatlimab.

In some embodiments, the monotherapy is performed with an-anti PD-L1 inhibitor, and in particular with anti-PD-L1 antibody.

In some embodiments, the bi-therapy is (as a combined therapy) is performed with anti-PD-L1 inhibitor and an-anti LAG-3 inhibitor, and in particular with anti-PD-L1 antibody and anti-LAG-3 antibody.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e.g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, 3-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, 3-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

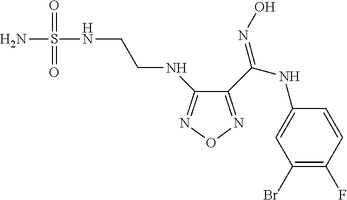 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

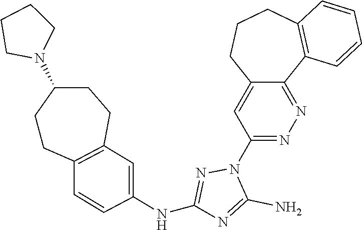 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In another embodiment, the invention is suitable to a method for predicting whether a subject suffering from melanoma will achieve a response to a monotherapy treatment.

In a particular embodiment, the invention relates to a method for determining whether a subject suffering from melanoma will achieve a response with an immune-checkpoint inhibitor comprising: i) quantifying the level of soluble CD27 (sCD27) in a biological sample obtained from the subject before a treatment with an immune-checkpoint inhibitor, ii) comparing the level of sCD27 quantified at step i) with its corresponding predetermined reference values and iii) concluding that the subject will not achieve a response to the treatment when the level of sCD27 is higher than its corresponding predetermined reference value or concluding that the subject will achieve a response to the treatment when the level of sCD27 is lower than its corresponding predetermined reference value.

In particular embodiment, the immune-checkpoint inhibitor is an anti-PD1 or an anti-PD-L1 and more particularly an anti-PD1 antibody or an anti-PD-L1 antibody.

In the context of the invention, inventors have shown that subjects treated with monotherapy with a high soluble CD27 concentration (>128.03 U/ml) had decreased survival compared to patients with a low soluble CD27 concentration (<128.03 U/ml) (p=0.00014).

Accordingly, in a second aspect the invention relates to a method for predicting the survival time of a subject suffering from melanoma comprising: i) determining the level of soluble CD27 (sCD27) in a biological sample; ii) comparing the level of sCD27 quantified at step i) with its corresponding predetermined reference value and iii) concluding that the subject will have a short survival time when the level of sCD27 quantified at step i) is higher than its corresponding predetermined reference value or concluding that the subject will have a long survival time when the level of sCD27 quantified at step i) is lower than its corresponding predetermined reference value.

As used herein, the term “predicting” means that the subject to be analyzed by the method of the invention is allocated either into the group of subjects who suffers or is susceptible to suffer from melanoma and/or metastatic melanoma, or into a group of subjects who does not suffer or is not suffering from.

As used herein, the terms “will achieve a response” or “respond” refer to the response to a treatment of the subject suffering from a cancer. Typically such treatment induces, ameliorates or otherwise causes an improvement in the pathological symptoms, disease progression or physiological conditions associated with or resistance to succumbing to a cancer.

In particular, in the context of the invention, the term “respond” refers to the ability of an immune checkpoint inhibitor to an improvement of the pathological symptoms, thus, the subject presents a clinical improvement compared to the subject who does not receive the treatment. The said subject is considered as a “responder” to the treatment. The term “not respond” refers to a subject who does not present any clinical improvement to the treatment with an immune checkpoint inhibitor treatment. This subject is considered as a “non-responder” or resistant to the treatment. Accordingly, the subject as considered “non-responder” has a particular monitoring in the therapeutic regimen. In a particular embodiment, the response to a treatment is determined by Response evaluation criteria in solid tumors (RECIST) criteria. This criteria refers to a set of published rules that define when tumors in cancer subjects improve (“respond”), stay the same (“stabilize”), or worsen (“progress”) during treatment. In the context of the invention, when the subject is identified as responder, it means that said subject improves overall and progression-free survival (OS/PFS). More particularly, the soluble CD27 is a tool to determine the overall survival (OS) of the subject before starting a treatment with an immune checkpoint inhibitor.

As used herein, the term “Overall survival (OS)” denotes the percentage of subject in a study or treatment group who are still alive for a certain period of time after they were diagnosed with or started treatment for a disease, such as a cancer (according to the invention).

In a particular embodiment, the method according to the invention further comprises a step of classification of subject by an algorithm and determining the level of soluble CD27.

Typically, the method of the present invention comprises a) quantifying the level of the soluble CD27 in the biological sample; b) implementing a classification algorithm on data comprising the quantified of sCD27 levels so as to obtain an algorithm output; c) determining the probability of the response of a subject suffering from melanoma to monotherapy or bi-therapy treatment.

In some embodiments, the method according to the invention wherein the algorithm is selected from Linear Discriminant Analysis (LDA), Topological Data Analysis (TDA), Neural Networks, Support Vector Machine (SVM) algorithm and Random Forests algorithm (RF). selected from Linear Discriminant Analysis (LDA), Topological Data Analysis (TDA), Neural Networks, Support Vector Machine (SVM) algorithm and Random Forests algorithm (RF).

In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

### Method for Treating Cancer and/or Metastatic Cancer

A method for treating a cancer and/or metastatic cancer in a subject in need thereof comprising a step of administering said subject with a therapeutically effective amount of an immune checkpoint inhibitor (monotherapy).

In a particular embodiment, the invention relates to a method for treating a cancer and/or metastatic cancer in a subject in need thereof comprising the following steps: i) determining the level of sCD27 according to the method as described above; ii) treating said subject with a therapeutically effective amount of an immune check point inhibitor when the level of sCD27 is lower than its predetermined reference value. Such treatment will be performed with an anti-PD-1 such as nivolumab.

In another embodiment, the invention relates to a method for treating a cancer and/or metastatic cancer in a subject in need thereof comprising the following steps: i) determining the level of sCD27 according to the method as described above; ii) treating said subject with a therapeutically effective amount of two immune check point inhibitors (bi-therapy) when the level of sCD27 is higher than its predetermined reference value.

In a particular embodiment, such treatment will be performed with an anti-PD-1 such as nivolumab and anti-CTLA-4 such as ipilimumab.

In a particular embodiment, such treatment will be performed with an anti-PD-1 such as nivolumab and anti-LAG-3 such as relatlimab.

The method according to the invention comprising: i) performing the method as described above, and ii) treating the subject with a monotherapy when the level of sCD27 quantified at step i) is lower than its corresponding predetermined reference value or treating with a bi-therapy when the level of sCD27 quantified at step i) is higher than its corresponding predetermined reference value.

The method for treating to the invention, wherein the immune checkpoint inhibitor is: an anti-PD-1 antibody, anti-PD-L1 antibody, anti-CTLA-4, anti-LAG-3 or anti-PD-L2 antibody.

The method for treating according to the invention wherein the monotherapy is performed with an anti-PD-1 antibody.

The method for treating according to the invention wherein the anti-PD-1 antibody is nivolumab.

The method for treating according to the invention, wherein two immune checkpoint inhibitors (bi-therapy) are administered as a combined preparation.

The method for treating according to the invention, wherein the bi-therapy is performed with an anti-PD-1 and anti-CTLA-4 antibodies.

The method for treating according to the invention, wherein the anti-PD-1 is nivolumab and the anti-CTLA-4 is ipilimumab.

The method according to the invention wherein the cancer and/or metastatic cancer is melanoma, kidney cancer, lung cancer or colorectal cancer.

The method for treating according to the invention, wherein the bi-therapy is performed with an anti-PD-1 and anti-LAG-3 antibodies.

The method for treating according to the invention, wherein the anti-PD-1 is nivolumab and the anti-LAG-3 is relatlimab. The method according to the invention wherein the cancer and/or metastatic cancer is defined above.

In a particular embodiment, the cancer is melanoma, kidney cancer, lung cancer or colorectal cancer.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” denotes a mammal, such as a rodent, a feline, a canine, and a primate. Particularly, the subject according to the invention is a human.

More particularly, the subject according to the invention has or is susceptible to have a cancer and/or metastatic cancer.

More particularly, the subject according to the invention has or is susceptible to have melanoma and/or metastatic melanoma.

More particularly, the subject according to the invention has or is susceptible to have kidney cancer and/or metastatic kidney cancer.

More particularly, the subject according to the invention has or is susceptible to have colorectal cancer and/or metastatic colorectal cancer.

In a particular embodiment, the subject according to the invention has or is susceptible to have a low level of sCD27 compared to its predetermined reference value. In this case, a monotherapy will be chosen such as a treatment with an anti-PD-1.

In a particular embodiment, the subject according to the invention has or is susceptible to have a high level of sCD27 compared to its predetermined reference value. In this case, a combined therapy (bi-therapy) will be chosen such as a treatment with an anti-PD-1 and anti-CTLA-4.

As used herein the terms “administering” or “administration” refer to the act of injecting or otherwise physically delivering a substance as it exists outside the body (e.g. an inhibitor of an immune check point) into the subject, such as by oral, mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a particular embodiment, the immune check point inhibitor is administered orally.

In another aspect, the invention relates to immune check point and ii) a classical treatment, as a combined preparation for use in the prevention and/or treatment of melanoma and/or metastatic melanoma in a subject in need thereof.

In a particular embodiment, the invention relates to i) an immune check point inhibitor and ii) a classical treatment for use by simultaneous, separate or sequential administration in the prevention and/or treatment of melanoma and/or metastatic melanoma in a subject in need thereof.

As used herein, the terms “combined treatment”, “combined therapy” or “therapy combination” refer to a treatment that uses more than one medication.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein, the term “classical treatment” refers to treatments well known in the art and used to treat cancer. In the context of the invention, the classical treatment refers to targeted therapy, radiation therapy, immunotherapy or chemotherapy.

In a particular embodiment, the invention relates to i) an immune check point inhibitor and ii) a radiation therapy used as a combined preparation for use in the prevention and/or treatment of melanoma and/or metastatic melanoma in a subject in need thereof.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the invention relates to i) an immune check point inhibitor and ii) a chemotherapy used as a combined preparation for use in the prevention and/or treatment of melanoma and/or metastatic melanoma in a subject in need thereof.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the invention relates to i) an immune check point inhibitor and ii) an anti-angiogenesis compound, as a combined preparation for use in the prevention and/or treatment of melanoma and/or metastatic melanoma in a subject in need thereof.

As used herein, the term “angiogenesis” refers to a physiological process involving the growth of new blood vessels from preexisting vessels. Angiogenesis is a combinatorial process that is regulated by a balance between pro- and anti-angiogenic molecules. Angiogenic stimuli (e.g. hypoxia or inflammatory cytokines) result in the induced expression and release of angiogenic growth factors such as vascular endothelial growth factor (VEGF) or fibroblast growth factor (FGF).

As used herein, the term “anti-angiogenesis” refers to any molecule which can inhibit angiogenesis that means the creation of new blood vessels. Typically, anti-angiogenesis compound is well known in the art and refers to the following compounds but not limited to bevacizumab (Avastin, anti-VEGF), itraconazole (anti-VGFR), carboxyamidotriazole, TNP-470 (an analog of fumagillin), CM101, IFN-α, IL-12, platelet factor-4, suramin, SU5416, thrombospondin, VEGFR antagonists, angiostatic steroids+heparin, Cartilage-Derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, αVβ3 inhibitors, linomide, ramucirumab, tasquinimod, ranibizumab, sorafenib (Nexavar), sunitinib (Sutent), pazopanib (Votrient), everolimus (Afinitor), cabozantinib.

By a “therapeutically effective amount” is meant a sufficient amount of an immune check point inhibitor for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition

The immune check point inhibitor alone and their combination or combination with a classical treatment as described above may be combined with pharmaceutically acceptable excipients, and optionally sustained-release matrices, such as biodegradable polymers, to form pharmaceutical compositions.

Accordingly, in another aspect, the invention relates to a pharmaceutical composition comprising immune check point inhibitor and pharmaceutically acceptable excipients.

In a particular embodiment, the pharmaceutical composition according to the invention comprising i) an immune check point inhibitor and ii) a classical treatment for use by simultaneous, separate or sequential administration in the prevention and/or treatment of cancer and/or metastatic cancer in a subject in need thereof.

In a particular embodiment, the pharmaceutical composition according to the invention comprising an ati-PD-1 and pharmaceutically acceptable excipients.

In a particular embodiment, the pharmaceutical composition according to the invention comprising an ati-PD-1, anti-CTLA-4 as a combined preparation and pharmaceutically acceptable excipients.

In a particular embodiment, the pharmaceutical composition according to the invention comprising an ati-PD-1, anti-LAG-3 as a combined preparation and pharmaceutically acceptable excipients.

In a particular embodiment, the pharmaceutical composition according to the invention is susceptible to treat cancer and/or metastatic cancer. Such cancer is defined above.

In a particular embodiment, the pharmaceutical composition according to the invention is susceptible to treat melanoma, kidney cancer, colorectal cancer and/or metastatic cancer.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Kits or Devices of the Present Invention

In another aspect, the present invention relates to a kit or device for performing the method of the present invention, comprising means for determining the level of the soluble CD27 in a biological sample.

In some embodiments, the kit or device comprises at least one binding partner (e.g. antibody or aptamer) specific for the soluble CD27 (immobilized or not on a solid support as described above). In some embodiments, the kit or device can include a second binding partner (e.g. antibody or aptamer) of the present invention which produces a detectable signal. Examples of kits include but are not limited to ELISA assay kits, and kits comprising test strips and dipsticks.

In some embodiments, the kit or device of the present invention further comprises a microprocessor to implement an algorithm on data comprising the level of soluble CD27 in the sample so as to determine the probability of responding to an immune checkpoint inhibitor. In some embodiments, the kit or device of the present invention further comprises a visual display and/or audible signal that indicates the probability determined by the microprocessor.

In some embodiments, the kit or device of the present invention comprises:
a mass spectrometer;
a receptacle into which the biological sample is placed, and which is connectable to the mass spectrometer so that the mass spectrometer can quantify the level of soluble CD27 in the sample;
a microprocessor to implement an algorithm on data comprising the levels of soluble CD27 in the sample so as to determine the probability of responding to an immune checkpoint inhibitor;
a visual display and/or audible signal that indicates the probability determined by the microprocessor.

### FIGURES

FIG. 1: sCD27 levels differentially predict survival in melanoma patients treated with single or combined therapy

Plasma sCD27 levels were measured in the Predimel cohort (A) composed of 78 melanoma patients treated with anti-PD-1 in monotherapy and 69 patients treated with anti-PD-1 and anti-CTLA-4 combined therapy (B) as well as in the Melbase cohort consisting of 102 patients treated with monotherapy (C) and 108 patients treated with combined therapy (D). The Youden Index and its associated cut-off was selected to define the optimized cut-off value (128.039 U/ml). The log-rank (Mantel-Cox) test was used to determine the P value. Patient numbers at risk at certain time points are presented. Values of P<0.05 were considered statistically significant.

FIG. 2: Previously defined cut-off value for sCD27 in RCC also differentially predict overall survival in melanoma patients treated by mono or combined immunotherapy

Plasma sCD27 concentrations were measured in the Predimel cohort (A) consisting of 78 melanoma patients treated with anti-PD-1 monotherapy and 69 patients treated with anti-PD-1 and anti-CTLA-4 dual therapy (B) as well as in the Melbase cohort consisting of 102 patients treated with monotherapy (C) and 108 patients treated with dual therapy (D). A previously defined cut-off of 112.7 U/ml in a kidney cancer cohort treated with anti-PD-1 (monotherapy) was selected. The log-rank (Mantel-Cox) test was used to determine the P value of the overall survival. Patient numbers at risk at certain time points are presented. Values of P<0.05 were considered statistically significant.

FIG. 3: sCD27 concentrations differentially predict progression-free survival in melanoma patients treated with mono- or combined immunotherapy

Plasma sCD27 levels were measured in the Predimel cohort (A) consisting of 78 melanoma patients treated with anti-PD-1 monotherapy and 69 patients treated with anti-PD-1 and anti-CTLA-4 dual therapy (B) as well as in the Melbase cohort consisting of 102 patients treated with monotherapy (C) and 108 patients treated with dual therapy (D). A previously defined cut-off of 112.7 U/ml in a kidney cancer cohort treated with anti-PD-1 (monotherapy) was selected. The log-rank (Mantel-Cox) test was used to determine the P value of the progression free survival (PFS). Patient numbers at risk at certain time points are presented. Values of P<0.05 were considered statistically significant.

FIG. 4: Correlation between plasmatic concentrations of sCD27 and clinical and biological parameters in the two melanoma cohorts

A For the Predimel cohort, a correlation matrix was performed between plasma concentrations of sCD27 and age as well as with different biological parameters (TMB and TMB syn (non-synonym mutations), PD-L1 expressed by tumour cells, serum LDH concentrations). The colour scale of positivity is shown on the right of the figure (Red=maximum correlation).

B For the Melbase cohort, plasma levels of soluble CD27 were compared with gender, presence or absence of Braf mutations, ECOG performance status and AJCC IV stage Mlc.

FIG. 5: sCD27 did not predict clinical response based on overall survival in RCC patients treated with the anti-PD-1 and anti-CTLA-4 combination therapy whatever the cut-off

sCD27 assays were performed prior to treatment with anti-PD-1 and anti-CTLA-4 in 81 patients with clear cell renal cancer. Different cut-offs were selected: either at a previously published cut-off (112.7 U/ml) in kidney cancer patients treated with anti-PD-1 [30] or at the optimised cut-off selected in melanoma patients (128.039 U/ml)

The log-rank (Mantel-Cox) test was used to determine the P value of the overall survival. HR with 95% Cis and patient numbers at risk at certain time points are presented. Values of P<0.05 were considered statistically significant.

### EXAMPLE 1

### Material & Methods

### Melanoma Cohorts

The Predimel cohort coordinated by Celeste Lebbe (Hôpital Saint Louis) has been registered at ClinicalTrial.gov (NCT02938728) and EudraCT (2016èAA00927-44). It was funded by PHRC-INCA. 147 patients have been included in this cohort. 78 of them was treated by the anti-PD-1 monotherapy and 69 of them by the anti-PD-1 and anti-CTLA-4 combination. Characteristics of the patients are described in Table 1 and 2. 12 deaths were observed in this cohort of which 7 were treated with monotherapy and 5 with the combination. 86 events were observed (85 progressions including 1 death without progression) of which 48 events among the 78 patients treated with monotherapy and 38 events among the 69 patients treated with the combined therapy. The PFS was achieved in 5.8 months in the overall population. The PFS rate at 12 months for patients treated with anti-PD-1 alone was 40.9% and 44.1% for patients treated with the combined therapy.

The Melbase Cohort is a National Cohort of Melanoma Stage IV and Unresectable Stage III Patients (ClinicalTrials.gov Identifier: NCT02828202). It is also coordinated by Celeste Lebbe. 210 patents have been included in this cohort. 102 of them was treated by the anti-PD-1 monotherapy and 108 of them by the anti-PD-1 and anti-CTLA-4 combination. Characteristics of the patients are described in Table 3 and 4. In this cohort, there were 46 deaths in patients with combination therapy (42.6%) and 61 deaths in patients with monotherapy alone (59.8%). In the Melbase cohort, 59 patients progressed and 34 died without progressing (53.3%). In patients on monotherapy 35 progressions (34.3%) were observed and in patients on combined therapy 24 progressions (22.2%) were recorded

### Renal Cancer Cohort

Bionikk Cohort for metastatic renal cell carcinoma treated by anti-PD-1 (nivolumab) and anti-CTLA-4 (ipilimumab)

The “BIONIKK” cohort [1], used as a validation cohort, is composed of patients with mRCC treated with first-line anti-PD-1 and anti-CTLA-4 (n=81) according to molecular classification into four randomized groups and for whom a plasma sample collected before treatment was available. This protocol was previously approved by the Ile de France ethics committee 8 (ref. 16.10.69) and registered at ClinicalTrials.gov under the NCT number 02960906.

The clinical results of this protocol have been recently published.

Plasma sCD27 Concentration Measurement by ELISA

The plasma sCD27 concentrations were determined with the CD27 (Soluble) Human Instant ELISA Kit (ThermoFisher Scientific, Massachusetts, United States) according to the manufacturer's instructions. sCD27 levels were measured in duplicate. Data were acquired with an MRX Revelation Microplate Reader (DYNEX Technologies, Virginia, United States).

### Statistical Analysis

For survival analyses, Kaplan-Meier plots were generated, and significant differences were identified using the log-rank Mantel-Cox test. A p value <0.05 was considered statistically significant.

### Results

Correlation Between Pre-Treatment sCD27 Levels and Overall Survival in Patients Treated with Anti-PD-1 Monotherapy or Anti-PD-1 and Anti-CTLA-4 Combined Therapy

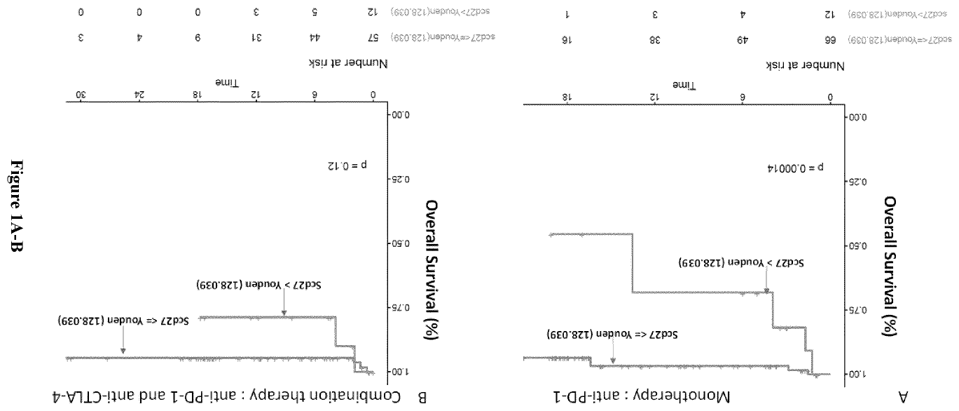

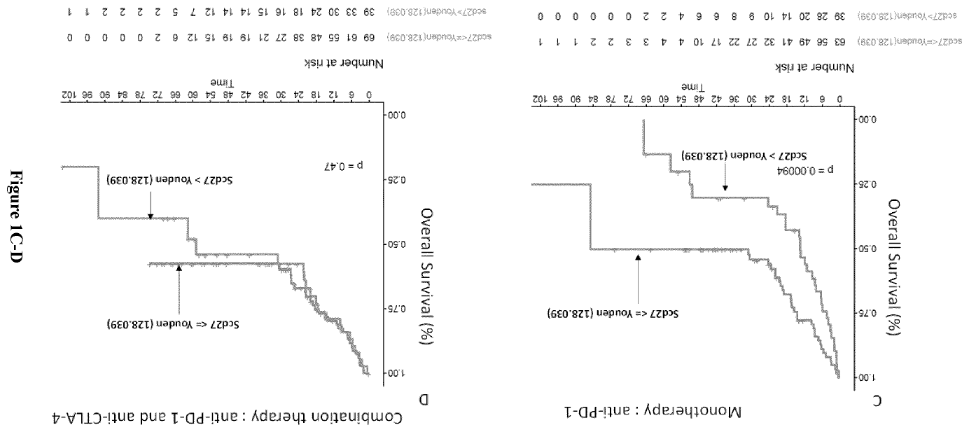

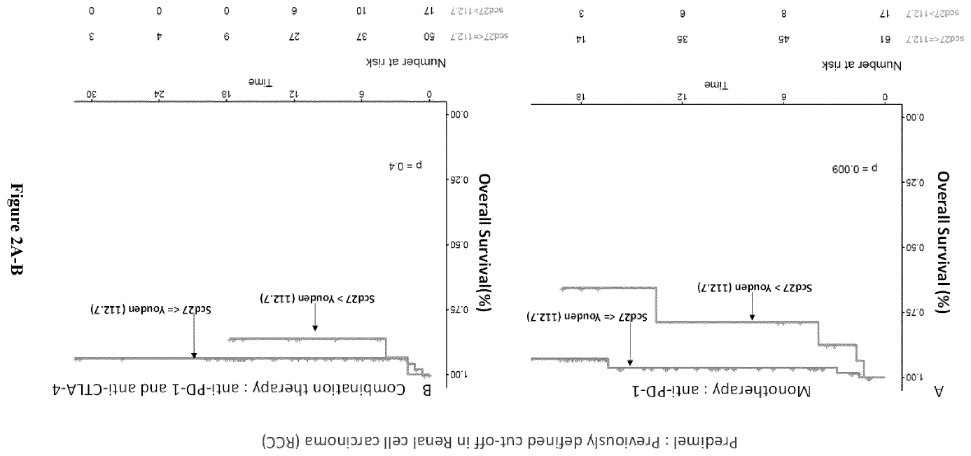

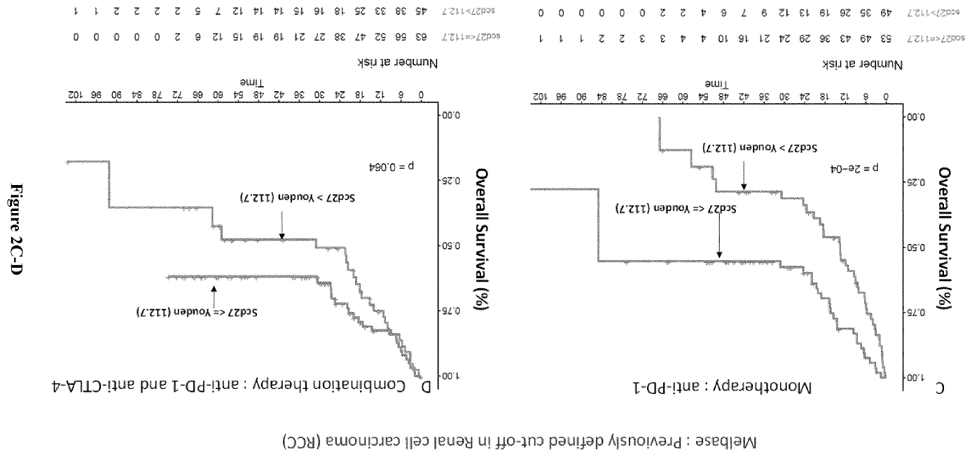

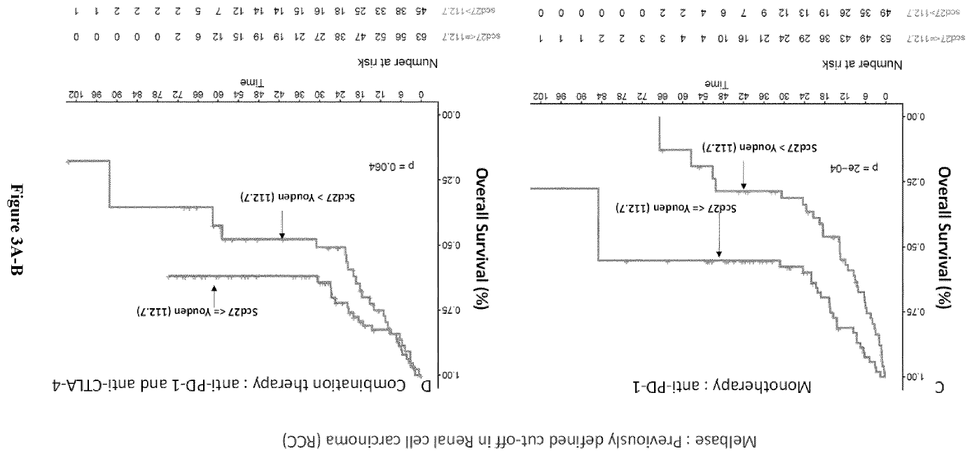

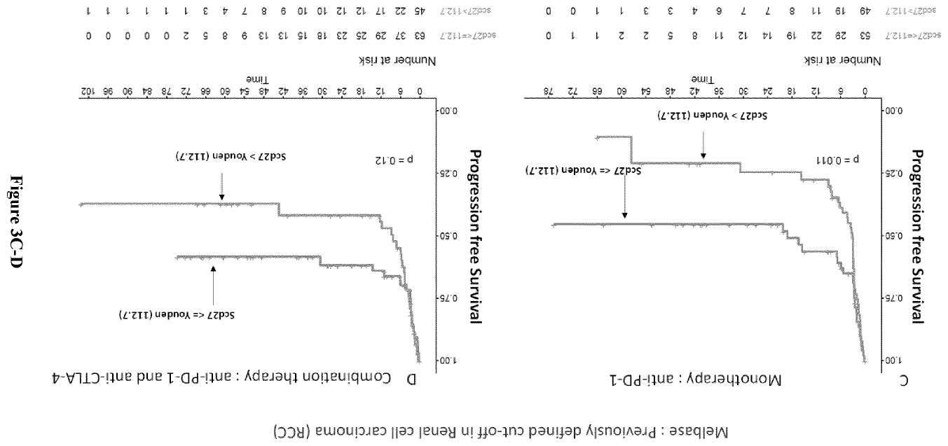

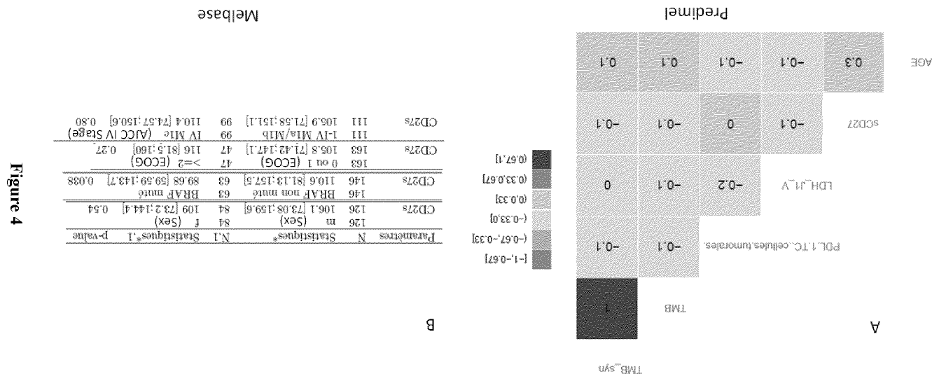

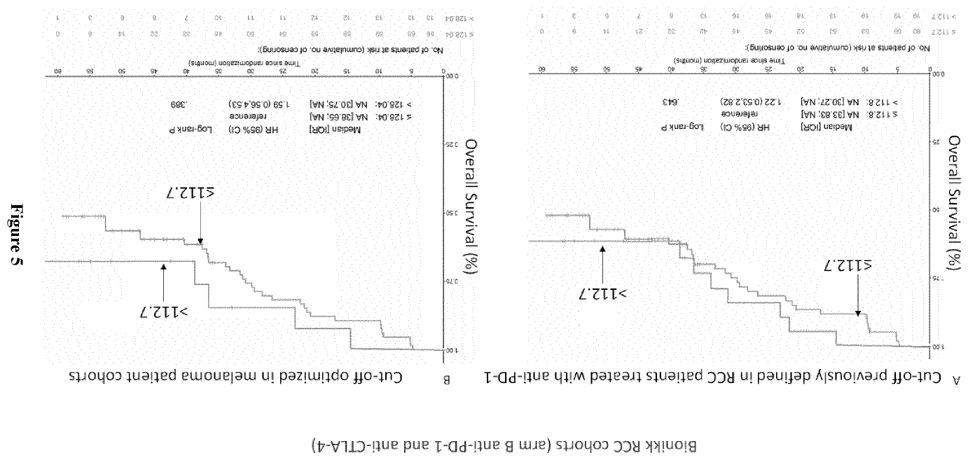

sCD27 was measured in all 147 patients in the Predimel cohort prior to treatment with monotherapy or combined therapy. Using an optimised cut-off, patients treated with monotherapy with a high soluble CD27 concentration (>128.03 U/ml) had decreased survival compared to patients with a low soluble CD27 concentration (<128.03 U/ml) (p=0.00014) (FIG. 1 A-B). In patients treated with monotherapy and still alive, sCD27 concentrations were 70.27 U/ml, while sCD27 concentrations were 153.1 U/ml in patients who died.

In contrast, in the group of patients treated with combined therapy, with the same optimised cut-off as in patients treated with monotherapy, sCD27 concentrations did not correlate significantly with survival (p=0.12) (FIG. 1). In patients who died in this group of patients treated with combined therapy, sCD27 concentrations were 68.4 U/ml vs 81.83 U/ml in living patients (FIG. 1i).

These results were confirmed in the Melbase cohort. Indeed, with the same optimised cut-off (128.03 U/ml) used in the Predimel cohort, patients treated with monotherapy with a high sCD27 concentration before treatment had a decreased survival compared to patients with a sCD27 concentration <128.03 U/ml (FIG. 1C). On the contrary, sCD27 concentrations are not correlated with survival in patients treated with combined therapy (FIG. 1D).

An impact of sCD27 concentrations on survival in kidney cancer patients treated with anti-PD-1 was previously reported by our team but using another optimal threshold (112.7 U/ml) [30]. We wanted to check if this threshold could also be used in our 2 cohorts of melanoma patients

In the Predimel cohort, we show that this threshold of 112.7 U/ml predicts the clinical course of patients treated with anti-PD-1. Thus, patients with high sCD27 concentrations have a decreased survival compared to patients with low concentrations (FIG. 2A)(p=0.009). We did not find a correlation with survival when dichotomising patients treated with combined therapy with this cut-off (FIG. 2B)(p=0.4). Similar results were found in the Melbase cohort with this cut-off of 112.7 U/ml predicting survival in patients treated with monotherapy (FIG. 2C) (p=0.0002) but not with combined therapy (FIG. 2D) (p=0.64).

Correlation Between Pre-Treatment sCD27 Levels and Progression-Free Survival in Patients Treated with Anti-PD-1 Monotherapy or Anti-PD-1 and Anti-CTLA-4 Combined Therapy

Using the same cut-off of 112.7 U/ml already used in kidney cancer patients, we observe that patients in the Predimel cohort with sCD27 concentrations higher than this threshold have a decreased progression-free survival compared to patients with low sCD27 concentrations (P=0.047 (HR 1.01-2.45))(FIG. 3A). In contrast, using the same cut-off, sCD27 levels did not predict PFS in patients treated with combined therapy (FIG. 3B)(p=0.7). These results were confirmed in the Melbase cohort, in which patients treated with monotherapy with sCD27 concentrations >112.7 U/ml had a decreased progression-free survival compared to patients with low sCD27 concentrations (FIG. 3C) (p=0.011). In patients treated with combined therapy, PFS was not associated with sCD27 levels (FIG. 3D)(P=0.12).

sCD27 Levels are not Correlated with Other Biological and Clinical Parameters that have an Impact on the Clinical Course of Patients

In the Predimel cohort, a correlation matrix including different biological parameters (LDH, TMB, PD-L1 expression by tumour cells) as well as patient age shows that sCD27 levels are not associated with these parameters (FIG. 4A). Furthermore, sCD27 levels were not correlated with patient gender, treatment lines, presence of brain or liver metastases, number of metastatic sites or AJCC stageIV of patients (results not shown).

In the Melbase cohort, sCD27 levels did not correlate with patient gender, Braf status, patient AJCC stage IV or ECOG score (FIG. 4B). Furthermore, sCD27 levels did not correlate with patient age (results not shown). Other biological parameters were not available in this cohort.

Soluble sCD27 Levels in Kidney Cancer Patients Treated with Dual Therapy do not Predict Clinical Outcome

In a previous study we showed that in the Bionikk cohort, anti-PD-1 treated kidney cancer patients with sCD27 levels >112.7 U/ml had decreased survival and resistance to immunotherapy {Benhamouda, 2022 #10374}. In the same cohort, we did not analyse the impact of sCD27 levels in patients treated with combined therapy. Using the same optimal cut-off of 112.7 U/ml defined in the previous work, there was no difference in survival between patients with soluble CD27 concentrations above or below this threshold (p=0.64)(HR 0.53-2.82)(FIG. 5A). Similar results were also observed using the optimised threshold of 128.04 U/ml defined in the 2 melanoma cohorts (p=0.38)(HR: 0.56-4.53) (FIG. 5B).

This work has shown that plasma levels of soluble CD27 are inversely correlated with survival in melanoma patients treated with anti-PD-1. This result was validated in 2 independent cohorts of patients representing 180 patients in total (FIGS. 1 and 2). It thus extends to melanoma the results that our team had found in kidney cancer on the role of sCD27 as a marker of resistance to anti-PD-1[30]. Interestingly, this predicting role of sCD27 in anti-PD-1 treated melanoma patients was found both with an optimal cut-off defined by the Youden test, but also with the same cut-off previously defined in kidney cancer. This suggests some analytical robustness of this marker. We have also shown that plasma sCD27 levels prior to anti-PD-1 treatment are inversely correlated with progression free survival (PFS) (FIG. 3). It does not appear that sCD27 levels are associated with different biological or clinical parameters suggesting that there is no confounding factor that could explain this result.

On the contrary, in patients with metastatic melanoma treated with anti-PD-1 and anti-CTLA-4, plasma sCD27 levels are not statistically significantly associated with patient survival or progression-free survival (FIG. 1-2-3). We therefore find a discrepancy between the predictive impact of sCD27 on survival of patients treated with anti-PD-1 immunotherapy alone or with combined anti-PD-1 and anti-CTLA-4 immunotherapy.

To validate this discrepancy in another tumour type, we showed that in kidney cancer patients treated with anti-PD-1 and anti-CTLA-4, the prognostic impact of sCD27 levels on survival was no longer observed (FIG. 5). These latest results suggest that in at least two cancers, plasma sCD27 levels may represent a biomarker of resistance to monotherapy by anti-PD-1, but not combined therapy with anti-PD-1 and anti-CTLA-4, a feature which could help to guide treatment choices. The various biomarker studies rarely distinguish between the different immunotherapies whose mechanisms of action may be different and lead to different biomarkers.

The current standard of care for patients with metastatic melanoma, especially if there are brain metastases, is the combination of anti-PD-1 and anti-CTLA-4.

However, this combination is more toxic than anti-PD-1 monotherapy. Various studies have shown that anti-PD-1 is more likely to act peripherally at the tumour site, while anti-CTLA-4 targets T cells in the lymph nodes first. In agreement with these results, it seems that anti-PD-1 has an action essentially on the reactivation of the T cells of the TME [48,49], while anti-CTLA-4 and its combination with anti-PD-1 would activate new anti-tumour effector T cells in the lymph nodes which would then migrate into the tumour [50]. This differential mechanism of action of monotherapy vs. combined therapy would explain the results observed in our work, because if sCD27 reflects intratumoral T cell dysfunction, it is expected that the immunotherapy combination would be less dependent on these intratumoral T cells. Consistent with this interpretation of the results, it was shown that CD8+ T cell infiltration or an intratumoral IFNγ signature was associated with a clinical response to anti-PD-(L)1, but not to the combination therapy [6]. Expression of HLA class II molecules on tumour cells is associated with a IFNγ signature in the TME—the IFN□ regulating HLA class II molecules—and correlates with response to anti-PD-(L)1, but not to anti-CTLA-4 [51]. In contrast, with anti-PD-1 and anti-CTLA-4 therapies, the presence of HEVs that promote lymphocyte entry into the tumour is necessary for clinical response. For anti-PD-(L)1 monotherapy, these vessels appear to be less required for treatment efficacy [52].

Combination therapy with anti-PD-1 and anti-CTLA-4 is now indicated in many cancers (melanoma, kidney cancer, lung cancer, malignant pleural mesothelioma, MSI high colorectal cancer, etc.). It appears to be more effective than anti-PD-1 monotherapy, but more toxic. To be able to guide patients between these 2 therapeutic options would meet a medical need. Plasma sCD27 concentrations represents a new type of biomarker to help in the management of these patients.

### EXAMPLE 2

To eliminate bias secondary to parameter imbalance that may be associated with treatment response between patients treated with monotherapy (anti-PD-1) vs. dual therapy (anti-PD-1 and anti-CTLA-4), we performed a propensity test on the Melbase cohort that balances the different variables potentially important in treatment response (mutated Braf, liver metastases, LDH, brain metastases, ECOG, age), simulating a stratified clinical trial for these parameters. This propensity test shows that sCD27, dichotomized with a threshold of 100, does indeed differentially predict response to monotherapy versus dual therapy, both in terms of overall survival (Table 1: interaction p=0.004) and clinical benefit (CR, PR and SD>6 months) as assessed by RECIST criteria (Table 2: interaction==0.009).

This propensity test therefore confirms that sCD27 predicts response to anti-PD-1 but not to anti-PD-1+anti-CTLA-4 in patients with metastatic melanoma who are balanced for various confounding factors.

TABLE 1
Cox model for overall survival adjusted by propensity score

| Parametres | N | Nevent | HR | IC95% | pvalue |
|---|---|---|---|---|---|
| Combi | 210 | 107 | 1.64 | (0.783; 3.436) | 0.19 |
| sCD27 > 100 | 210 | 107 | 4.592 | (2.365; 8.918) | <0.0001 |
| interaction | 210 | 107 | 0.254 | (0.099; 0.652) | 0.004 |

In the Melbase cohort (n=210 patients with 107 deaths, we balanced the 2 groups of patients treated with monotherapy (anti-PD-1) or dual therapy (anti-PD-1 and anti-CTLA-4) for the different variables (mutated Braf, liver metastases, LDH, brain metastases, ECOG, age) using a propensity score. The p-value of the interaction shows that the differential predictive value of sCD27 for patients treated with monotherapy versus dual therapy remains significant for overall survival.

TABLE 2
Logistic model for (Complete response (CR), Partial response
(PR) or Stable Disease (SD) adjusted by propensity score

| Parametres | N | Nevent | OR | IC95% | p-value |
|---|---|---|---|---|---|
| combi | 179 | 120 | 0.81 | (0.28-2.33) | 0.70 |
| sCD27 > 100 | 179 | 120 | 0.22 | (0.08-0.63) | 0.005 |
| interaction | | | 7.1 | (1.65-30.53) | 0.009 |

In the Melbase cohort, we balanced the 2 groups of patients treated with monotherapy (anti-PD-1) or dual therapy (anti-PD-1 and anti-CTLA-4) for the different variables (mutated Braf, liver metastases, LDH, brain metastases, ECOG, age) using a propensity score. The p-value of the interaction shows that the differential predictive value of sCD27 for patients treated with monotherapy versus dual therapy remains significant for clinical response based on Recist criteria and including complete response (CR), partial response (PR) and clinical benefit (Stability of the disease for more than 6 months).

### REFERENCES

Throughout this application, various references describe the state of the art to which this invention pertains. The disclosures of these references are hereby incorporated by reference into the present disclosure.
1 Garbe C, Amaral T, Peris K, Hauschild A, Arenberger P, Basset-Seguin N et al. European consensus-based interdisciplinary guideline for melanoma. Part 2: Treatment—Update 2022. Eur J Cancer 2022; 170: 256-284.
2 Auslander N, Zhang G, Lee J S, Frederick D T, Miao B, Moll T et al. Robust prediction of response to immune checkpoint blockade therapy in metastatic melanoma. Nat Med 2018; 24: 1545-1549.
3 Gide T N, Quek C, Menzies A M, Tasker A T, Shang P, Holst J et al. Distinct Immune Cell Populations Define Response to Anti-PD-1 Monotherapy and Anti-PD-1/Anti-CTLA-4 Combined Therapy. Cancer Cell 2019; 35: 238-255 e236.
4 Miller B C, Sen D R, Al Abosy R, Bi K, Virkud Y V, LaFleur M W et al. Subsets of exhausted CD8(+) T cells differentially mediate tumor control and respond to checkpoint blockade. Nat Immunol 2019; 20: 326-336.
5 Sade-Feldman M, Yizhak K, Bjorgaard S L, Ray J P, de Boer C G, Jenkins R W et al. Defining T Cell States Associated with Response to Checkpoint Immunotherapy in Melanoma. Cell 2018; 175: 998-1013 e1020.
6 Li F, Li C, Cai X, Xie Z, Zhou L, Cheng B et al. The association between CD8+ tumor-infiltrating lymphocytes and the clinical outcome of cancer immunotherapy: A systematic review and meta-analysis. EClinicalMedicine 2021; 41: 101134.
7 Scott A C, Dundar F, Zumbo P, Chandran S S, Klebanoff C A, Shakiba M et al. TOX is a critical regulator of tumour-specific T cell differentiation. Nature 2019; 571: 270-274.
8 Newell F, Pires da Silva I, Johansson P A, Menzies A M, Wilmott J S, Addala V et al. Multiomic profiling of checkpoint inhibitor-treated melanoma: Identifying predictors of response and resistance, and markers of biological discordance. Cancer Cell 2022; 40: 88-102 e107.
9 Reschke R, Yu J, Flood B, Higgs E F, Hatogai K, Gajewski T F. Immune cell and tumor cell-derived CXCL10 is indicative of immunotherapy response in metastatic melanoma. J Immunother Cancer 2021; 9.
10 Weber J, Del Vecchio M, Mandala M, Gogas H, Arance A, Dalle S et al. Adjuvant nivolumab (NIVO) versus ipilimumab (IPI) in resected stage III/I V melanoma: 3-year efficacy and biomarker results from the phase 3 CheckMate 238 trial. in ESMO. 2019. Barcelona, Spain: Annals of Oncology; 30 (Suppl 5): 2801
11 Reck M, Rodriguez-Abreu D, Robinson A G, Hui R, Csoszi T, Fulop A et al. Pembrolizumab versus Chemotherapy for P D-L1-Positive Non-Small-Cell Lung Cancer. N Engl J Med 2016.
12 Kluger H M, Zito C R, Turcu G, Baine M K, Zhang H, Adeniran A et al. P D-L1 Studies Across Tumor Types, Its Differential Expression and Predictive Value in Patients Treated with Immune Checkpoint Inhibitors. Clin Cancer Res 2017; 23: 4270-4279.
13 Giraldo N A, Nguyen P, Engle E L, Kaunitz G J, Cottrell T R, Berry S et al. Multidimensional, quantitative assessment of PD-1/P D-L1 expression in patients with Merkel cell carcinoma and association with response to pembrolizumab. J Immunother Cancer 2018; 6: 99.
14 Girault I, Adam J, Shen S, Roy S, Brard C, Faouzi S et al. A P D-1/P D-L1 Proximity Assay as a Theranostic Marker for PD-1 Blockade in Patients with Metastatic Melanoma. Clin Cancer Res 2022; 28: 518-525.
15 Cabrita R, Lauss M, Sanna A, Donia M, Skaarup Larsen M, Mitra S et al. Tertiary lymphoid structures improve immunotherapy and survival in melanoma. Nature 2020; 577: 561-565.
16 Voabil P, de Bruijn M, Roelofsen L M, Hendriks S H, Brokamp S, van den Braber M et al. An ex vivo tumor fragment platform to dissect response to PD-1 blockade in cancer. Nat Med 2021; 27: 1250-1261.
17 Martinet L, Le Guellec S, Filleron T, Lamant L, Meyer N, Rochaix P et al. High endothelial venules (HEVs) in human melanoma lesions: Major gateways for tumor-infiltrating lymphocytes. Oncoimmunology 2012; 1: 829-839.
18 Lynch K T, Young S J, Meneveau M O, Wages N A, Engelhard V H, Slingluff C L, Jr. et al. Heterogeneity in tertiary lymphoid structure B-cells correlates with patient survival in metastatic melanoma. J Immunother Cancer 2021; 9.
19 Schumacher T N, Schreiber R D.Neoantigens in cancer immunotherapy. Science 2015; 348: 69-74.
20 Shao X M, Huang J, Niknafs N, Balan A, Cherry C, White J et al. HLA class I I immunogenic mutation burden predicts response to immune checkpoint blockade. Ann Oncol 2022; 33: 728-738.
21 Hodi F S, Wolchok J D, Schadendorf D, Larkin J, Long G V, Qian X et al. TMB and Inflammatory Gene Expression Associated with Clinical Outcomes following Immunotherapy in Advanced Melanoma. Cancer Immunol Res 2021; 9: 1202-1213.
22 Spranger S, Bao R, Gajewski T F. Melanoma-intrinsic beta-catenin signalling prevents anti-tumour immunity. Nature 2015; 523: 231-235.
23 Peng W, Chen J Q, Liu C, Malu S, Creasy C, Tetzlaff M T et al. Loss of PTEN Promotes Resistance to T Cell-Mediated Immunotherapy. Cancer Discov 2016; 6: 202-216.
24 Davoli T, Uno H, Wooten E C, Elledge S J. Tumor aneuploidy correlates with markers of immune evasion and with reduced response to immunotherapy. Science 2017; 355.
25 Lee J H, Shklovskaya E, Lim S Y, Carlino M S, Menzies A M, Stewart A et al. Transcriptional downregulation of MHC class I and melanoma de-differentiation in resistance to PD-1 inhibition. Nat Commun 2020; 11: 1897.
26 Zhang Z, Wang Z X, Chen Y X, Wu H X, Yin L, Zhao Q et al. Integrated analysis of single-cell and bulk RNA sequencing data reveals a pan-cancer stemness signature predicting immunotherapy response. Genome Med 2022; 14: 45.
27 Montesion M, Murugesan K, Jin D X, Sharaf R, Sanchez N, Guria A et al. Somatic HLA Class I Loss Is a Widespread Mechanism of Immune Evasion Which Refines the Use of Tumor Mutational Burden as a Biomarker of Checkpoint Inhibitor Response. Cancer Discov 2021; 11: 282-292.
28 Zhou Z, Chen M M, Luo Y, Mojumdar K, Peng X, Chen H et al. Tumor-intrinsic SIRPA promotes sensitivity to checkpoint inhibition immunotherapy in melanoma. Cancer Cell 2022; 40: 1324-1340 e1328.
29 Tuomela K, Ambrose A R,Davis D M. Escaping Death: How Cancer Cells and Infected Cells Resist Cell-Mediated Cytotoxicity. Front Immunol 2022; 13: 867098.
30 Benhamouda N, Sam I, Epaillard N, Gey A, Phan L, Pham H P et al. Plasma CD27, a Surrogate of the Intratumoral CD27-CD70 Interaction, Correlates with Immunotherapy Resistance in Renal Cell Carcinoma. Clin Cancer Res 2022; 28: 4983-4994.
31 Rozeman E A, Hoefsmit E P, Reijers ILM, Saw RPM, Versluis J M, Krijgsman O et al. Survival and biomarker analyses from the OpACIN-neo and OpACIN neoadjuvant immunotherapy trials in stage III melanoma. Nat Med 2021; 27: 256-263.
32 Hugo W, Zaretsky J M, Sun L, Song C, Moreno B H, Hu-Lieskovan S et al. Genomic and Transcriptomic Features of Response to Anti-PD-1 Therapy in Metastatic Melanoma. Cell 2016; 165: 35-44.
33 Jiang P, Gu S, Pan D, Fu J, Sahu A, Hu X et al. Signatures of T cell dysfunction and exclusion predict cancer immunotherapy response. Nat Med 2018; 24: 1550-1558.
34 Xiong D, Wang Y, You M. A gene expression signature of TREM2(hi) macrophages and gammadelta T cells predicts immunotherapy response. Nat Commun 2020; 11: 5084.
35 Zhang Y, Vu T, Palmer D C, Kishton R J, Gong L, Huang J et al. A T cell resilience model associated with response to immunotherapy in multiple tumor types. Nat Med 2022; 28: 1421-1431.
36 Xiao X, Xu C, Yang W, Yu R. Inconsistent prediction capability of ImmuneCells. Sig across different RNA-seq datasets. Nat Commun 2021; 12: 4167.
37 Wolf Y, Samuels Y. Intratumor Heterogeneity and Antitumor Immunity Shape One Another Bidirectionally. Clin Cancer Res 2022; 28: 2994-3001.
38 Madore J, Vilain R E, Menzies A M, Kakavand H, Wilmott J S, Hyman J et al. P D-L1 expression in melanoma shows marked heterogeneity within and between patients: implications for anti-PD-1/P D-L1 clinical trials. Pigment Cell Melanoma Res 2015; 28: 245-253.
39 Obeid J M, Hu Y, Erdag G, Leick K M, Slingluff C L, Jr. The heterogeneity of tumor-infiltrating CD8+ T cells in metastatic melanoma distorts their quantification: how to manage heterogeneity?Melanoma Res 2017; 27: 211-217.
40 Lee J H J, Lyle M, Menzies A M, Chan M M K, Lo S, Clements A et al. Metastasis-specific patterns of response and progression with anti-PD-1 treatment in metastatic melanoma. Pigment Cell Melanoma Res 2018; 31: 404-410.
41 Ahmadzadeh M, Johnson L A, Heemskerk B, Wunderlich J R, Dudley M E, White D E et al. Tumor antigen-specific CD8 T cells infiltrating the tumor express high levels of PD-1 and are functionally impaired. Blood 2009; 114: 1537-1544.
42 Kruger S, Legenstein M L, Rosgen V, Haas M, Modest D P, Westphalen C B et al. Serum levels of soluble programmed death protein 1 (sPD-1) and soluble programmed death ligand 1 (sPD-L1) in advanced pancreatic cancer. Oncoimmunology 2017; 6: e1310358.
43 Forschner A, Battke F, Hadaschik D, Schulze M, Weissgraeber S, Han C T et al. Tumor mutation burden and circulating tumor DNA in combined CTLA-4 and PD-1 antibody therapy in metastatic melanoma—results of a prospective biomarker study. J Immunother Cancer 2019; 7: 180.
44 Long G V, Arance A, Mortier L, Lorigan P, Blank C, Mohr P et al. Antitumor activity of ipilimumab or BRAF+/−MEK inhibition after pembrolizumab treatment in patients with advanced melanoma: analysis from KEYNOTE-006. Ann Oncol 2022; 33: 204-215.
45 Machiraju D, Wiecken M, Lang N, Hulsmeyer I, Roth J, Schank T E et al. Soluble immune checkpoints and T-cell subsets in blood as biomarkers for resistance to immunotherapy in melanoma patients. Oncoimmunology 2021; 10: 1926762.
46 Turiello R, Capone M, Giannarelli D, Morretta E, Monti M C, Madonna G et al. Serum CD73 is a prognostic factor in patients with metastatic melanoma and is associated with response to anti-PD-1 therapy. J Immunother Cancer 2020; 8.
47 Pich C, Sarrabayrouse G, Teiti I, Mariame B, Rochaix P, Lamant L et al. Melanoma-expressed CD70 is involved in invasion and metastasis. Br J Cancer 2016; 114: 63-70.
48 Inozume T, Hanada K, Wang Q J, Ahmadzadeh M, Wunderlich J R, Rosenberg S A et al. Selection of CD8+PD-1+ lymphocytes in fresh human melanomas enriches for tumor-reactive T cells. J Immunother 2010; 33: 956-964.
49 Wei S C, Levine J H, Cogdill A P, Zhao Y, Anang N A S, Andrews M C et al. Distinct Cellular Mechanisms Underlie Anti-CTLA-4 and Anti-PD-1 Checkpoint Blockade. Cell 2017; 170: 1120-1133 e1117.
50 Walunas T L, Lenschow D J, Bakker C Y, Linsley P S, Freeman G J, Green J M et al. CTLA-4 can function as a negative regulator of T cell activation. Immunity 1994; 1: 405-413.
51 Rodig S J, Gusenleitner D, Jackson D G, Gjini E, Giobbie-Hurder A, Jin C et al. MHC proteins confer differential sensitivity to CTLA-4 and PD-1 blockade in untreated metastatic melanoma. Sci Transl Med 2018; 10.
52 Asrir A, Tardiveau C, Coudert J, Laffont R, Blanchard L, Bellard E et al. Tumor-associated high endothelial venules mediate lymphocyte entry into tumors and predict response to PD-1 plus CTLA-4 combination immunotherapy. Cancer Cell 2022; 40: 318-334 e319.

1-17. (canceled)

18. A method for treating a cancer and/or a metastatic cancer in a subject in need thereof comprising a step of
quantifying a level of soluble CD27 (sCD27) in a biological sample obtained from the subject before a treatment with a monotherapy or bi-therapy,
determining that the level of sCD27 is lower than a reference value, and treating the subject having a lower sCD27 level with a therapeutically effective amount of a monotherapy; or
determining that the level of sCD27 is higher than the reference value and treating the subject having a higher sCD27 level with a therapeutically effective amount of a bi-therapy.

19. The method of claim 18, wherein the monotherapy and/or the bi-therapy comprises administration of at least one immune check point inhibitor.

20. The method of claim 19, wherein the at least one immune checkpoint inhibitor is: an anti-PD-1 antibody, an anti-PD-L1 antibody, an anti-CTLA-4, an anti-LAG-3 or an anti-PD-L2 antibody.

21. The method according to claim 18, wherein the monotherapy is performed with an anti-PD-1 antibody.

22. The method according to claim 21, wherein the anti-PD-1 antibody is nivolumab.

23. The method according to claim 18, wherein a the bi-therapy is performed with two immune checkpoint inhibitors.

24. The method according to claim 23, wherein the two immune checkpoint inhibitors are administered as a combined preparation.

25. The method according to claim 23, wherein the two immune checkpoint inhibitors are an anti-PD-1 antibody and an anti-CTLA-4 antibody.

26. The method according to claim 25, wherein the anti-PD-1 antibody is nivolumab and the anti-CTLA-4 is ipilimumab.

27. The method according to claim 23, wherein the two immune checkpoint inhibitors are anti-PD-1 antibody and anti-LAG-3 antibody.

28. The method according to claim 27, wherein the anti-PD-1 antibody is nivolumab and the anti-LAG-3 antibody is relatlimab.

29. The method according to claim 18, wherein the cancer and/or metastatic cancer is melanoma, kidney cancer, lung cancer or colorectal cancer.
